(19) United States
(12) Patent Application Publication
(10) Pub. No.: US 2025/0356492 A1
(43) Pub. Date: Nov. 20, 2025

(54) Systems and Methods for Quantifying Skin Pigmentation Conditions

(71) Applicant: ImageD, LLC, Clinton Township, MI (US)

(72) Inventors: Iltefat Husain Hamzavi, Northville, MI (US); Indermeet Kohli, Westland, MI (US)

(21) Appl. No.: 19/212,066

(22) Filed: May 19, 2025

Related U.S. Application Data
(60) Provisional application No. 63/649,934, filed on May 20, 2024.

(51) Int. Cl. G06T 7/00; G16H 50/20

(52) U.S. Cl. CPC G06T 7/0014; G16H 50/20; G06T 2207/10024; G06T 2207/20081; G06T 2207/30088

(57) ABSTRACT

The present disclosure relates to systems and methods for quantifying hypo- or hyper-skin pigmentation conditions. An example method includes providing an image of a skin surface. The method also includes selecting a plurality of color channels from among a plurality of color models. The method yet also includes forming a color-adjusted version of the image based on the selected combination of color channels. The method additionally includes extracting a mask based on the color-adjusted version of the image. The method yet further includes determining, based on the extracted mask, a normal portion of the skin surface. The method also includes determining, based on the extracted mask, a differently-pigmented portion of the skin surface. The method additionally includes providing information indicative of the differently-pigmented portion of the skin surface.

### CROSS-REFERENCE TO RELATED APPLICATION

The present application claims the benefit of U.S. Patent Application No. 63/649,934, filed May 20, 2024, the content of which is herewith incorporated by reference.

### BACKGROUND

Vitiligo is a skin condition characterized by patches of the skin losing their pigment. The diagnosis and monitoring of vitiligo has traditionally been based primarily on clinical examination by a dermatologist, often supported by a Wood's lamp (e.g., ultraviolet light) examination or skin biopsy. However, these methods have limitations.

Firstly, the subjective nature of visual examination can lead to inconsistencies in diagnosis, particularly in the early stages of the disease or in cases with atypical presentation. Secondly, while Wood's lamp examination enhances the contrast between vitiliginous and normal skin, it requires specific lighting conditions and expert interpretation. Skin biopsy, although definitive, is invasive and not practical for monitoring disease progression.

In recent years, there have been attempts to develop more objective and quantifiable methods for diagnosing and monitoring vitiligo. These efforts include various imaging techniques and computer-aided analysis. However, these methods often lack precision, are time-consuming, and/or require expensive equipment, limiting their widespread use in clinical settings.

Accordingly, there remains a need for an improved approach to the diagnosis and quantification of vitiligo that is accurate, efficient, non-invasive, and user-friendly, both for clinicians and for patient self-monitoring.

### SUMMARY

The present invention relates generally to the field of dermatological diagnosis and analysis. More specifically, it pertains to a novel system and method for the diagnosis and quantification of vitiligo.

In embodiments, the advanced techniques that use feature selection methods, machine learning algorithms, and image processing techniques to automate this process. Key features from color spaces such as HSV, LAB, and RGB have been extracted. Also, a clustering algorithm has been employed to segment vitiligo regions. Our preliminary results have been encouraging, revealing noticeable improvement over the manual techniques previously used.

In a first aspect, a system is provided. The system includes a controller having at least one processor and a memory. The memory stores program instructions that are executable by the at least one processor so as to carry out operations. The operations include receiving an image of a skin surface. The operations also include selecting a combination of color channels from among a plurality of color models. The operations additionally include forming a color-adjusted version of the image based on the selected combination of color channels. The operations yet further include extracting a mask based on the color-adjusted version of the image. The operations also include determining, based on the extracted mask, a normal portion of the skin surface. The operation yet further includes determining, based on the extracted mask, a differently-pigmented portion of the skin surface. The operations include providing information indicative of the differently-pigmented portion of the skin surface.

In a second aspect, a method includes providing an image of a skin surface. The method also includes selecting a combination of color channels from among a plurality of color models. The method yet further includes forming a color-adjusted version of the image based on the selected combination of color channels. The method additionally includes extracting a mask based on the color-adjusted version of the image. The method also includes determining, based on the extracted mask, a normal portion of the skin surface. The method additionally includes determining, based on the extracted mask, a differently-pigmented portion of the skin surface. Furthermore, the method includes providing information indicative of the differently-pigmented portion of the skin surface.

Other aspects and applications are possible and contemplated within the scope of the present disclosure.

### BRIEF DESCRIPTION OF THE FIGURES

FIG. 1 illustrates a system, according to an example embodiment.

FIG. 2 illustrates an image of a skin surface that includes a depigmented portion, according to an example embodiment.

FIG. 3 illustrates an original image of a skin surface that includes a depigmented portion, according to an example embodiment.

FIG. 4 illustrates various color channels from a plurality of color models of the original image, according to an example embodiment.

FIG. 5 illustrates a color-adjusted version of the original image, according to an example embodiment.

FIG. 6 illustrates a method, according to an example embodiment.

FIG. 7 illustrates a displayed screen of a user interface, according to an example embodiment.

FIG. 8 illustrates a displayed screen of a user interface, according to an example embodiment.

FIG. 9 illustrates a displayed screen of a user interface, according to an example embodiment.

FIG. 10 illustrates a displayed screen of a user interface, according to an example embodiment.

FIG. 11 illustrates a displayed screen of a user interface, according to an example embodiment.

FIG. 12 illustrates a displayed screen of a user interface, according to an example embodiment.

### DETAILED DESCRIPTION

Example methods, devices, and systems are described herein. It should be understood that the words “example” and “exemplary” are used herein to mean “serving as an example, instance, or illustration.” Any embodiment or feature described herein as being an “example” or “exemplary” is not necessarily to be construed as preferred or advantageous over other embodiments or features. Other embodiments can be utilized, and other changes can be made, without departing from the scope of the subject matter presented herein.

Thus, the example embodiments described herein are not meant to be limiting. Aspects of the present disclosure, as generally described herein, and illustrated in the figures, can be arranged, substituted, combined, separated, and designed in a wide variety of different configurations, all of which are contemplated herein.

Further, unless context suggests otherwise, the features illustrated in each of the figures may be used in combination with one another. Thus, the figures should be generally viewed as component aspects of one or more overall embodiments, with the understanding that not all illustrated features are necessary for each embodiment.

### 1. Overview

Systems and methods described herein may provide benefits over traditional techniques to diagnose and monitor hypopigmentation and hyperpigmentation conditions. For example, the described imaging and determination using a machine learning model may be substantially faster and more uniform than approaches that rely on manual and sometimes tedious “fingertip area measurements.” Even using more modern photographic imaging techniques, recent methods rely on manually selecting blue channels and adjusting thresholds to extract features, a process that is laborious and subject to variation.

Hypopigmentation in skin refers to conditions where parts of the skin become lighter or completely white, usually because the cells that produce melanin (melanocytes) are absent or cease to function properly. The most well-known condition associated with skin depigmentation is vitiligo. Vitiligo is characterized by the loss of skin color in patches and can affect any area of the body, including the hair and inside of the mouth. The exact cause of vitiligo is not known, but it is believed to be an autoimmune condition where the immune system attacks and destroys the melanocytes in the skin. Other conditions that may cause depigmentation include: albinism, a genetic condition characterized by a lack of melanin, resulting in very light skin, hair, and eyes; piebaldism: a rare genetic condition that manifests as a white patch of skin (often on the forehead) and white hair (poliosis) in the affected area; and post-inflammatory hypopigmentation, lighter skin patches that occur after an injury or skin inflammation, such as eczema, psoriasis, or acne.

Hyperpigmentation is a skin condition characterized by dark patches or spots on the skin that are darker than the surrounding areas. This condition occurs when an excess of melanin, the brown pigment that produces normal skin color, forms deposits in the skin. Hyperpigmentation can occur in small patches, cover large areas, or affect the entire body. Hyperpigmentation is often caused by: sun exposure (increased melanin production to protect the skin from UV rays); hormonal influences, such as those seen in pregnancy or with conditions like melasma or chloasma; certain medications including some chemotherapy drugs can cause hyperpigmentation as a side effect; inflammation and skin injuries including those related to acne vulgaris; and medical conditions (some underlying health conditions may also cause hyperpigmentation). Melasma, post-inflammatory hyperpigmentation (PIH), and solar lentigines (age spots, sun spots) are specific types of hyperpigmentation, each with its own set of causes and characteristics. Treatment options vary depending on the cause and may include topical treatments, laser therapy, and preventive measures to avoid worsening of the condition.

Embodiments described herein include use feature selection methods, machine learning algorithms, and image processing techniques to automate the process of obtaining appropriate settings for identifying hypopigmented or hyperpigmented regions of skin. In some examples, key features can be extracted from image color spaces such as HSV, LAB, and RGB. Additionally, a clustering algorithm can be utilized to segment regions of skin that have become hypopigmented or hyperpigmented.

After the image capture process, the images can be processed in numerous different ways. Image adjustments such as cropping, rotation, white balance, exposure, tone, hue, color, contrast, and/or brightness are possible and contemplated. Additionally or alternatively, selection of an appropriate color space is important so as to accurately and robustly analyze skin conditions like vitiligo.

For image analysis, especially in the context of pigmentation-related skin conditions like vitiligo, the choice of color space plays an important role in the accuracy and robustness of the results. Traditional RGB space, while being ubiquitous, does not consistently capture the nuances and variability of skin tones and conditions. By exploring alternative color spaces such as HSV (Hue, Saturation, Value) and LAB (Lightness, a: green to magenta, b: blue to yellow), a more discriminative or intuitive understanding of skin variations can be obtained.

By exploring diverse color spaces and feature extraction parameters, higher sensitivity and specificity has been obtained to detect vitiligo regions. The present disclosure describes systems and methods that utilize these various color spaces and other techniques to enhance the efficacy of clustering and analysis techniques.

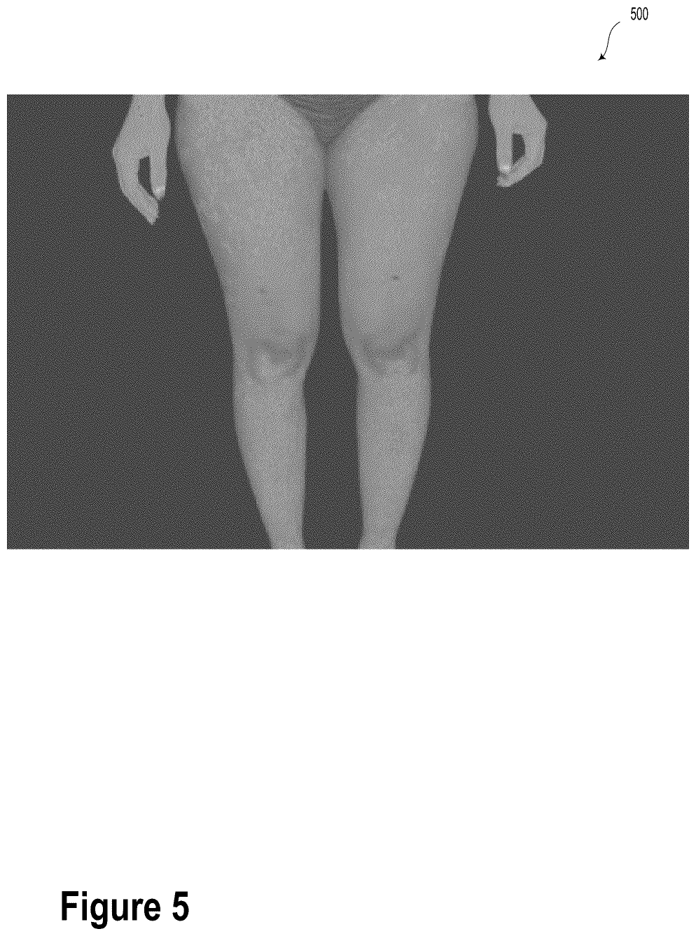

For vitiligo or other skin pigmentation-based conditions, a new color space may be formed by selecting channels from traditional color spaces, which can include (RGB-B, HSV-V, Lab-b). Using this combination of color channels from among a plurality of traditional color models, a color-adjusted version of the image can be formed. In such scenarios, the vitiligo affected skin (orange, FIG. 5) is clearly distinguishable from normal skin tone (blueish, FIG. 5), making it easier to cluster the region with accuracy.

Using unsupervised machine learning techniques, different regions of the frame can be clustered based on color variations of the pixels. A mask of the depigmented (or hyperpigmented) region can be extracted by selecting a region of interest (e.g., a desired cluster).

The present disclosure provides visual examples to demonstrate the effectiveness of the new method compared to traditional techniques. It also includes images processed through ImageJ (adjusted) and original images, showing the improvements in clustering and feature extraction.

Examples embodiments describe systems and methods for vitiligo detection that utilize advanced color feature extraction and unsupervised machine learning for more accurate and efficient segmentation. This represents a significant improvement over manual and traditional methods, offering potential for enhanced diagnostic and treatment strategies in dermatology.

ImageJ is a public domain, Java-based image processing program developed at the National Institutes of Health (NIH). The program is widely used for scientific image analysis and can handle a variety of image formats. ImageJ provides features like image editing, analysis, processing, and display, and it supports a range of processing tasks, including statistical analysis of image data, geometric transformations, and color manipulation. Its extensible nature, with the ability to run plugins, ImageJ allows for specialized functionalities to be added, making it a versatile tool in fields like biology, medical imaging, and neuroscience. While certain embodiments described herein utilize ImageJ, it will be understood that other image processing software programs are possible and contemplated within the scope of the present disclosure.

### Imaging Technique

The image capture process may include capturing a plurality of digital images of skin surfaces using, for example, a digital single lens reflex (DSLR) camera or another type of high-resolution camera. In some example embodiments, a camera of a mobile device (e.g., smartphone, tablet, etc.) may be utilized to capture the images described herein. In some embodiments, cross-polarized images could be utilized for input images. In such scenarios, the image capture system could include a polarizer optically coupled to the camera and an illuminator (e.g., a flash or continuous light source) oriented perpendicular to the polarizer to provide capture of cross polarized images. It will be understood that other image capture devices are possible and contemplated within the scope of the present disclosure. In various embodiments, external lighting (e.g., flash, ring light, etc.) may be utilized to provide uniform lighting of the skin surface.

### User Interface and Output

In some embodiments, a user interface could include a live-view display and/or a viewfinder configured to provide a view or display of the field of view of the image capture device. Additionally or alternatively, the user interface could include selectable image capture and/or image adjustment options, such as white balance, ISO, image capture details, among other possibilities. In some examples, data visualization may be provided by the user interface. In various embodiments, data visualizations could include, in some embodiments, a histogram of tonal distribution of the scene. Additionally or alternatively, the data visualization could include a Vitiligo Area Scoring Index (VASI) score as described herein. Other aspects of the systems and methods described herein could be displayed via the user interface. In some examples, various functions and/or selections described herein could be provided by a user via a touchscreen, a selector wheel, a button, or another type of pointing or selection device via the user interface.

In some examples, the user interface may be configured to receive information about the subject (e.g., name, age, gender, description of skin condition, etc.) and provide an interface to upload captured images of the subject to one or more local or remote (e.g., cloud) file folders. Stored images may then be processed as described above. The output files may include images with differently depigmented areas as being superimposed over a clinical image with normal color model. Objective quantitative information, such as the area of differently pigmented skin in mm² is also provided. Such quantitative skin area information can be attained by applying a sticker of known size on the subject's skin. In such scenarios, the sticker could be captured in each input image and subsequent scaling of images could be based on the known size of the sticker.

### Data Collection and Handling

Image data captured by the image capture device could be stored on a local, distributed, and/or cloud-based file storage system. The image data could be provided in one or more typical image file formats. In some examples, the image data could be encrypted, password-protected, and/or otherwise access-controlled.

### Practical Applications

In some examples, the captured images could be used to document an area of the body surface impacted by differently pigmented skin. This information could be captured and measured over several subject visits and thus help assess changes (e.g., increasing or decreasing skin pigmentation area and/or another change in patient condition). It will be understood that the systems and methods described herein could be configured for and/or designed so as to be used in a clinical or at-home setting. Furthermore, in some embodiments, the image capture system could be located in a clinical and/or at-home setting while other aspects of the systems and methods described herein could be located in a different location or located at multiple other locations.

### 2. Example Systems

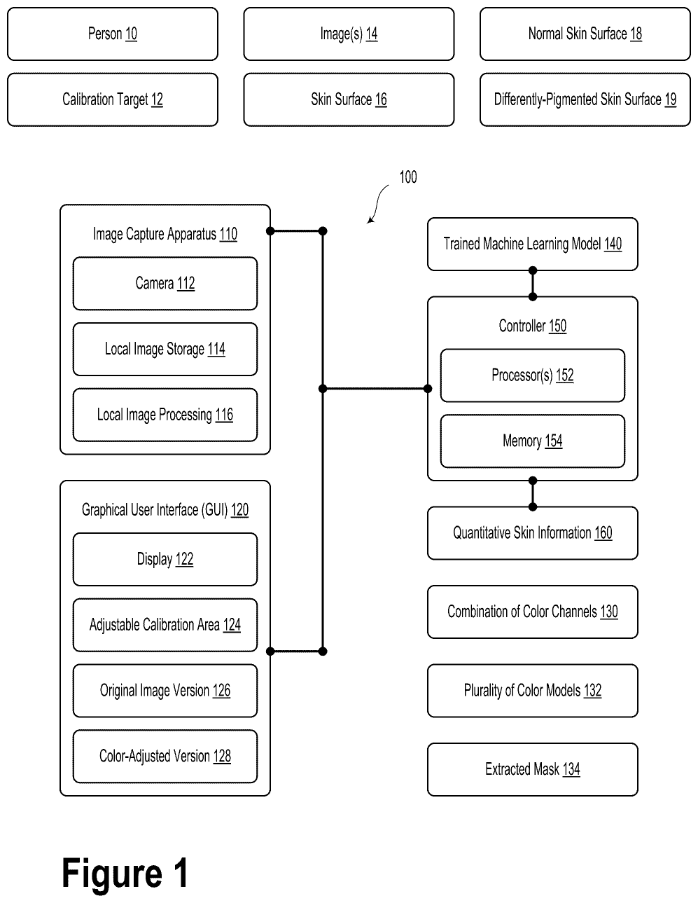

FIG. 1 illustrates a system 100, according to an example embodiment. System 100 includes a controller having at least one processor and a memory. In such scenarios, the memory stores program instructions that are executable by the at least one processor so as to carry out operations.

The operations include receiving an image 14 of a skin surface 16. The operations also include selecting a combination of color channels 130 from among a plurality of color models 132. The operations additionally include forming a color-adjusted version 128 of the image based on the selected combination of color channels 130.

The operations also include extracting a mask 134 based on the color-adjusted version of the image 128. The operations additionally include determining, based on the extracted mask 134, a normal portion of the skin surface 18. The operations include determining, based on the extracted mask 134, a differently-pigmented portion of the skin surface 19. Yet further, the operations include providing information indicative of the differently-pigmented portion of the skin surface 19.

In various examples, the selected combination of color channels 130 includes RGB-B, HSV-V, and Lab-b*.

In some examples, the selected combination of color channels 130 are selected from a plurality of color models 132. The plurality of color models 132 includes at least one of RGB (Red, Green, Blue), CMYK (Cyan, Magenta, Yellow, Black), HSV (Hue, Saturation, Value), HSL (Hue, Saturation, Lightness), Lab (Lightness, a*, b*), Lab Color (CIELAB), or XYZ (CIE 1931 Color Space).

In various embodiments, extracting the mask 134 could include clustering one or more regions of the color-adjusted version of the image 128 so as to form regions of interest.

In example embodiments, extracting the mask 134 could include utilizing an unsupervised machine learning technique based on color variations of pixels of the color-adjusted version of the image 128.

In various examples, extracting the mask 134 could include utilizing a trained machine learning model 140 based on color variations of pixels of the color-adjusted version of the image 128. In some examples, the trained machine learning model 140 could be trained with a plurality of training data images.

In some examples, providing information indicative of the differently-pigmented portion of the skin surface 19 includes providing a Vitiligo Area Scoring Index (VASI) score.

In various examples, the image of the skin surface includes a calibration target 12. In such scenarios, determining the normal portion of the skin surface 18 and determining the differently-pigmented portion of the skin surface 19 is based on an apparent size of the calibration target 12 within the image 14 of the skin surface.

In some examples, system 100 could include an image capture apparatus 110. In such scenarios, the operations also include causing the image capture apparatus 110 to capture the image 14 of the skin surface.

In some embodiments, system 100 may include a graphical user interface (GUI) 120. In such scenarios, the operations include displaying, via the GUI 120, an original version of the image 126 and the color-adjusted version of the image 128. In some examples, the operations may include displaying the information indicative of the differently-pigmented portion of the skin surface 19.

In various examples, the controller 150 could include a processor 152, which could include a microprocessor, a digital signal processor, a graphics processing unit (GPU), a tensor processing unit (TPU), or a central processing unit (CPU). Other types of computing devices are possible and contemplated, such as a field-programmable gate array (FPGA) or an application-specific integrated circuit (ASIC).

In various examples, the controller 150 could include a Bluetooth communication interface, a Wi-Fi communication interface, and a USB-Serial interface.

In various embodiments, the system 100 may include a graphical user interface (GUI) 170. The GUI 170 could include a display 172 and may be configured to display an adjustable calibration area 124, the original image version 126, and the color-adjusted version 128.

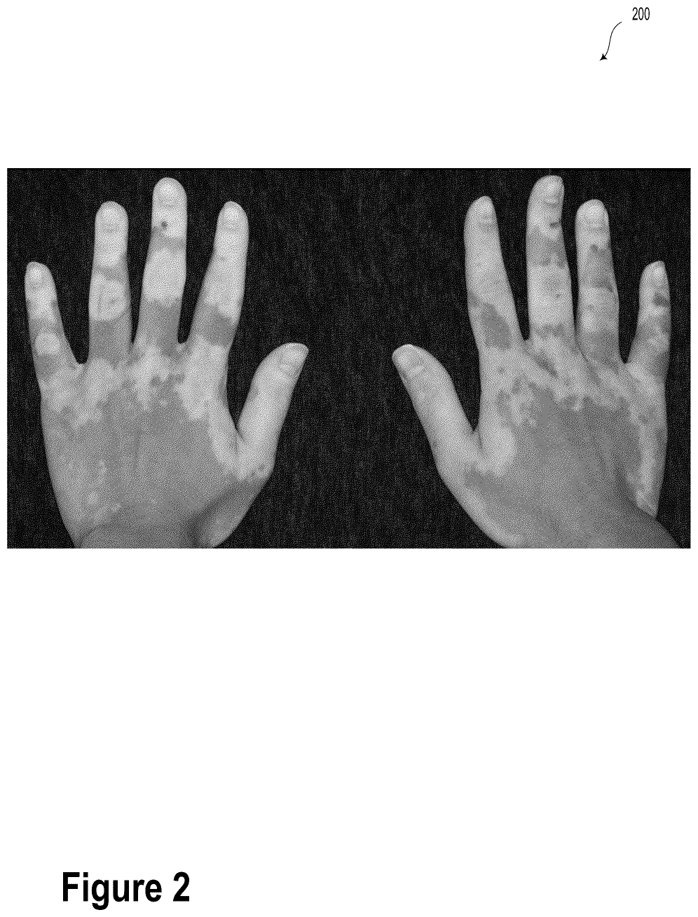

FIG. 2 illustrates an image of a skin surface 200 that includes a depigmented portion, according to an example embodiment.

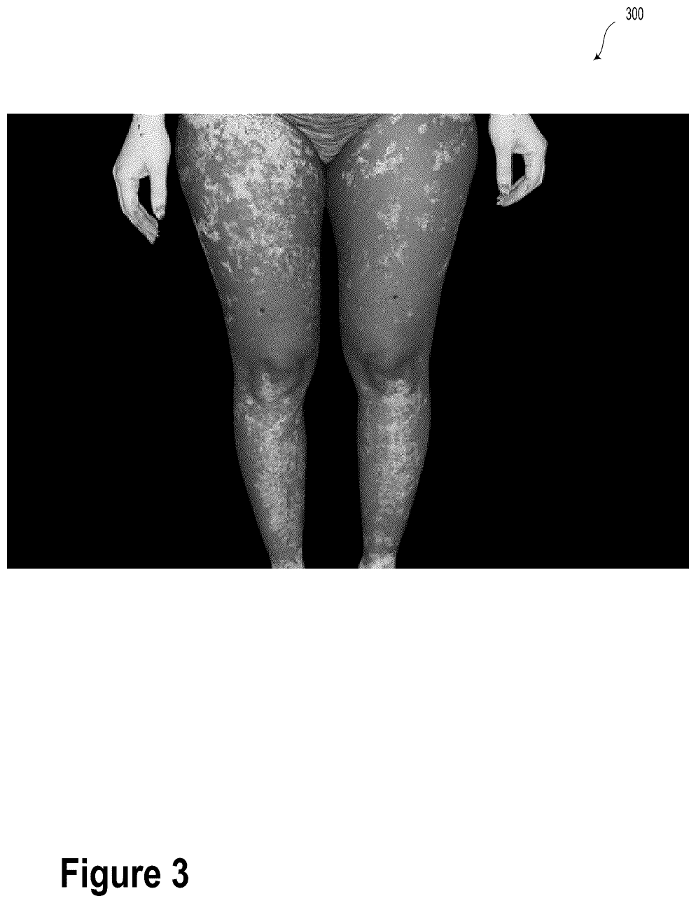

FIG. 3 illustrates an original image of a skin surface 300 that includes a depigmented portion, according to an example embodiment.

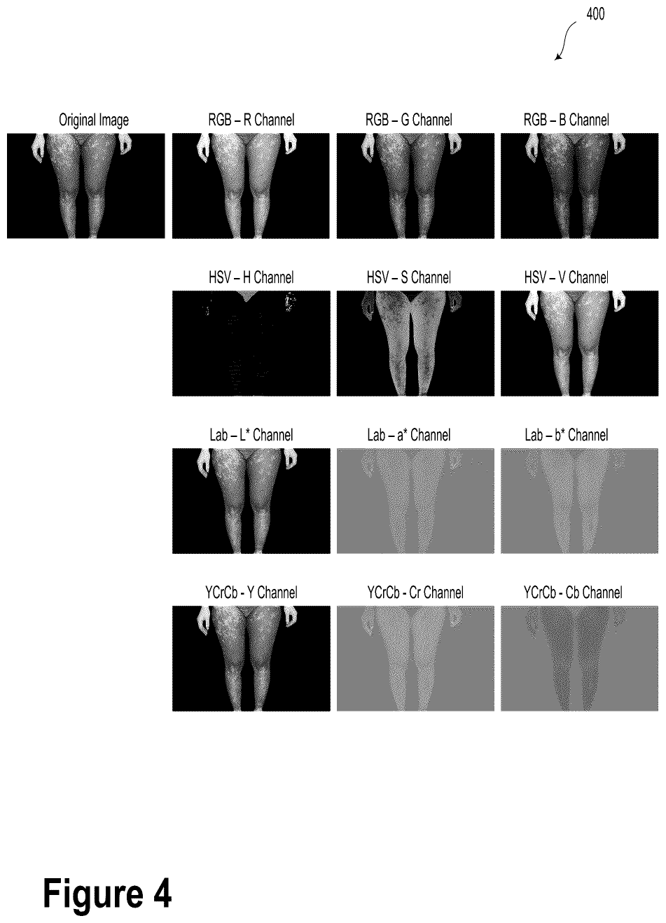

FIG. 4 illustrates various color channels 400 from a plurality of color models of the original image, according to an example embodiment.

FIG. 5 illustrates a color-adjusted version 500 of the original image, according to an example embodiment.

### 3. Methodology

Systems and methods described herein may utilize some or all of the following steps or blocks to calculate depigmented skin area.

Extract the mask of the depigmented area (e.g., depigmentation mask).

Count the total number of pixels in the depigmentation mask.

Extract a black dot sticker from the body part and measure its diameter. This normalizes differences due to camera position, zoom, and angle.

Obtain the pixel-to-meter ratio from the measured circle.

$$Lastly , calculate the depigmentation area using the formula :$$

$$Depigmented Area = \sum [ Pixels in Depigmentation ] \times {[ Pixel to Meter Ratio ]}^{2} ( unit {mm}^{2} ) .$$

The VASI score provides the percentage of total body area covered by a specific depigmented area. It accomplishes this by standardizing Finger Tip Units (FTUs) as a unit representing a specific percentage of the body.

First, we repeat the area calculation process for the fingertip.

$$Fingertip Area = \sum [ Pixels in Fingertip ] \times [ Pixel to Meter Ratio ] 2 {( unit mm )}^{2}$$

Next, the Intelligent VASI (i-VASI) score may be obtained using the formula:

$$\% Depigmented Body Part Surface = Normalized Depigmented Area / Finger Tip Area \times [ FTU to Body Percentage ]$$

Where “FTU to Body Percentage” is the standardized value. Its value is set as 0.03 which represents an approximate percentage of the fingertip compared to the total body surface.

At the time of image capture, a black calibration dot (e.g., a circular black sticker) should be affixed to a portion of normal skin in each image. Furthermore, a black background for the hand image should be used.

### 4. Example Methods

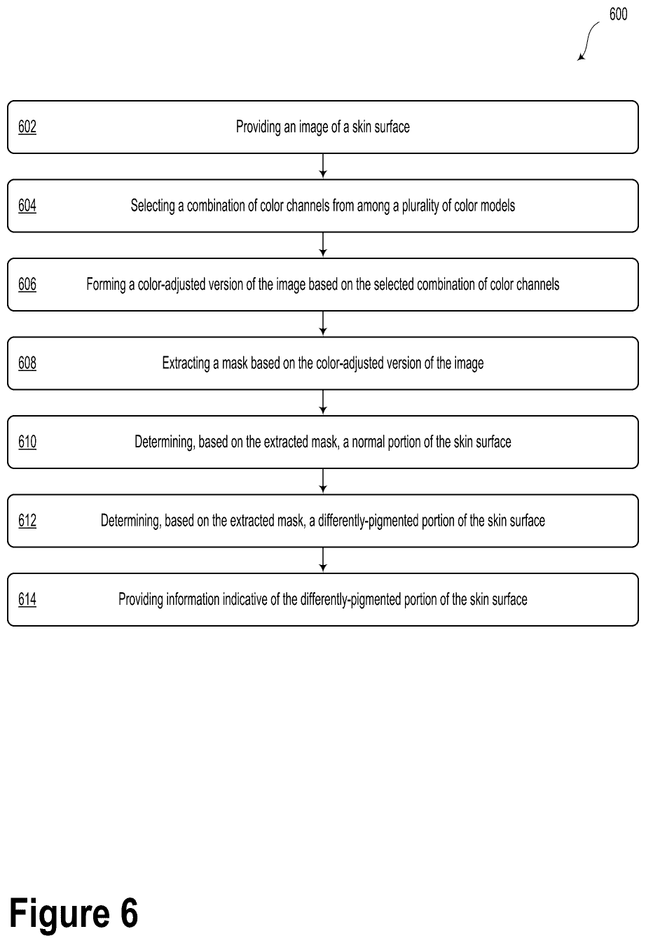

FIG. 6 illustrates a method 600, according to an example embodiment.

Method 600 may include fewer or more steps or blocks than those expressly illustrated or otherwise disclosed herein. Furthermore, respective steps or blocks of method 1000 may be performed in any order and each step or block may be performed one or more times. In some embodiments, some or all of the blocks or steps of method 600 may be carried out by controller 150 and/or other portions of system 100. In some examples, method 600 could use images of skin surfaces such as those illustrated in FIGS. 2 and 3. Furthermore, method 600 could isolate such images into various color channels (e.g., color channels 400) as illustrated in FIG. 4. Yet further, method 600 could provide a color-adjusted version (e.g., color-adjusted version 500) of an original image, such as that illustrated in FIG. 5.

Block 602 includes providing an image of a skin surface (e.g., image 14). Block 604 includes selecting a combination of color channels (e.g., combination of color channels 130) from among a plurality of color models (e.g., color models 132).

Block 606 includes forming a color-adjusted version of the image 128 based on the selected combination of color channels 130.

Block 608 also includes extracting a mask 134 based on the color-adjusted version of the image 128.

Block 610 includes determining, based on the extracted mask, a normal portion of the skin surface (normal skin surface 18).

Block 612 includes determining, based on the extracted mask, a differently-pigmented portion of the skin surface (e.g., pigmented skin surface 19).

Block 614 includes providing information (e.g., quantitative skin information 160) indicative of the differently-pigmented portion of the skin surface.

In various examples, the selected combination of color channels includes RGB-B, HSV-V, and Lab-b*.

In example embodiments, the selected combination of color channels are selected from a plurality of color models including at least one of: RGB (Red, Green, Blue), CMYK (Cyan, Magenta, Yellow, Black), HSV (Hue, Saturation, Value), HSL (Hue, Saturation, Lightness), Lab (Lightness, a*, b*), Lab Color (CIELAB), or XYZ (CIE 1931 Color Space).

In some embodiments, extracting the mask could include clustering one or more regions of the color-adjusted version of the image so as to form regions of interest.

In example embodiments, extracting the mask includes utilizing an unsupervised machine learning technique based on color variations of pixels of the color-adjusted version of the image.

In some examples, extracting the mask could include utilizing a trained machine learning model based on color variations of pixels of the color-adjusted version of the image.

In various examples, the trained machine learning model was trained with a plurality of training data images.

In various embodiments, providing information indicative of the differently-pigmented portion of the skin surface could include providing an intelligent Vitiligo Area Scoring Index (i-VASI) score.

In some scenarios, the image of the skin surface could include a calibration target. In such situations, determining the normal portion of the skin surface and determining the differently-pigmented portion of the skin surface could be based on an apparent size of the calibration target within the image of the skin surface.

### 5. Machine Learning

Some embodiments may use machine learning (ML) and/or artificial intelligence (AI) methods of analysis, including but not limited to one or more of: Convolutional Neural Networks (CNNs), Recurrent Neural Networks (RNNs), Generative Adversarial Networks (GANs), and Transfer Learning, where pre-trained models are fine-tuned for specific tasks. Additionally, Object Detection models like R-CNN, YOLO, and SSD could be utilized within the context of the present disclosure. In such scenarios, the object detection models could be specifically designed to identify and locate depigmented skin regions within images, balancing accuracy and processing speed for real-time applications.

Additionally or alternatively, the ML model could involve supervised learning. For example, a supervised learning model could be trained using labeled data, such as a dataset including a plurality of images of human skin. In such scenarios, the images could be associated with one or more labels, such as: 1) depigmented skin or normal skin; 2) a percentage of depigmented skin with respect to skin area; 3) demographic information about the subject; 4) body part imaged; and 5) other metadata related to image capture, among other possibilities. Such a supervised learning model could be used to classify new images and/or detect depigmented skin regions.

In various examples, a supervised ML model could learn from the input images and their corresponding labels through a process of optimization, gradually improving its ability to recognize patterns, features, and objects. A CNN could be utilized to automatically and iteratively learn the optimal features for the task. This approach is widely applied in facial recognition, medical imaging, and autonomous vehicles, demonstrating high accuracy and adaptability to various visual contexts—such as that of identifying and quantifying skin depigmentation.

In some embodiments, a time series analysis could be conducted to better understand the sequence and timing of changes in skin pigmentation of an individual or set of individuals. For instance, algorithms like Long Short-Term Memory (LSTM) networks could be utilized to identify very slight differences in skin coloration and automatically detect deviations. Additionally or alternatively, the results of these models may be beneficial for predicting future skin pigmentation trends based on historical change data, aiding in long-term therapeutic and skin care/management plans.

Within the scope of the present disclosure, unsupervised learning could be used to train the underlying machine learning models. Unsupervised learning is a type of machine learning approach that deals with data without labeled responses. In such examples, the model attempts to learn the patterns and the structure from the data (e.g., images of skin surfaces) without any guidance on what a desirable outcome might resemble. When it comes to training a machine learning model for image recognition of skin depigmentation (and/or hyperpigmentation), using unsupervised learning, the process focuses on identifying inherent groupings or patterns within the image data.

First, the model extracts features from the images. This involves analyzing the raw pixel data to identify patterns, colors, textures, shapes, or other relevant features within the images. Techniques such as principal component analysis (PCA) or autoencoders can be used for dimensionality reduction to highlight the most informative features in the images.

Once features are extracted, the model can apply clustering algorithms to group similar images (and/or groups of adjacent pixels) together. Clustering is the process of partitioning the dataset into groups (clusters), where images (and/or similar adjacent pixels) in the same cluster are more similar to each other than to those in other clusters. Common algorithms include:

K-means Clustering: Assigns images to clusters by minimizing the variance within each cluster. This method requires specifying the number of clusters in advance.

Hierarchical Clustering: Builds a tree of clusters by iteratively merging or splitting existing clusters, which can be useful for understanding the data's hierarchical structure.

DBSCAN (Density-Based Spatial Clustering of Applications with Noise): Groups together closely packed images, marking as outliers those that lie alone in low-density regions. This algorithm does not require specifying the number of clusters beforehand.

### 6. Example User Interfaces

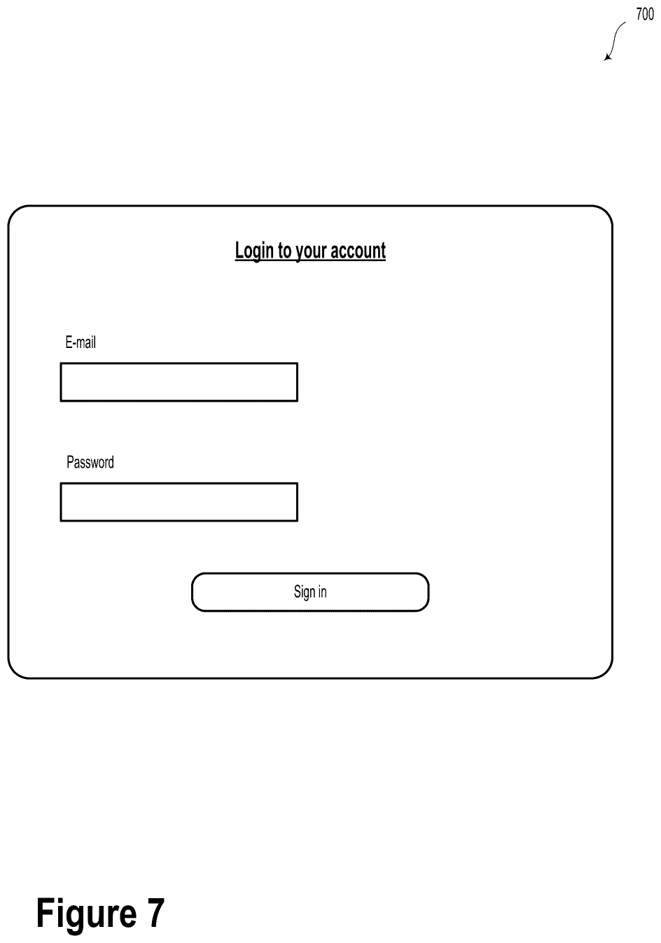

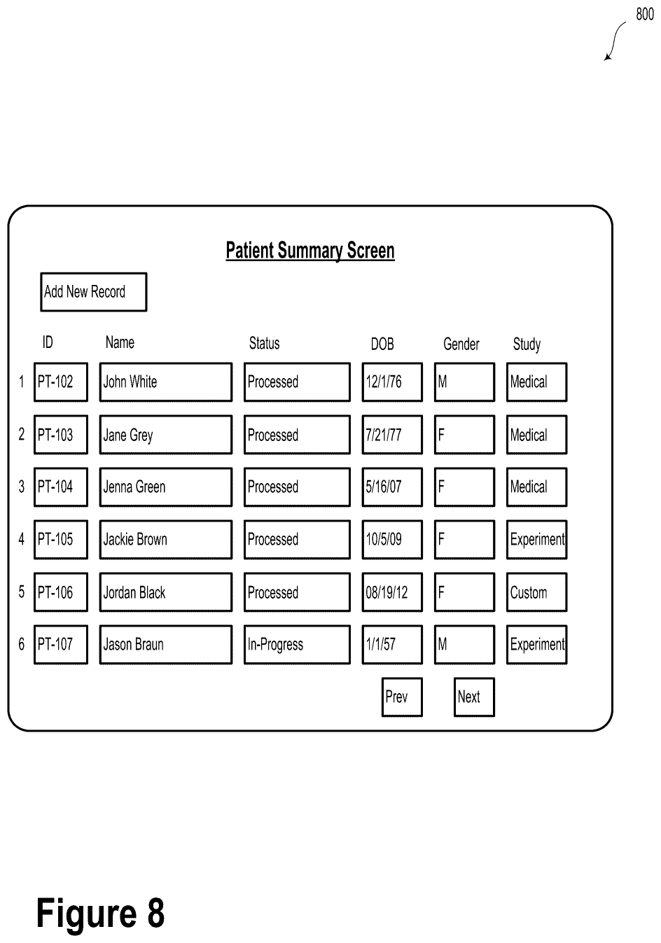

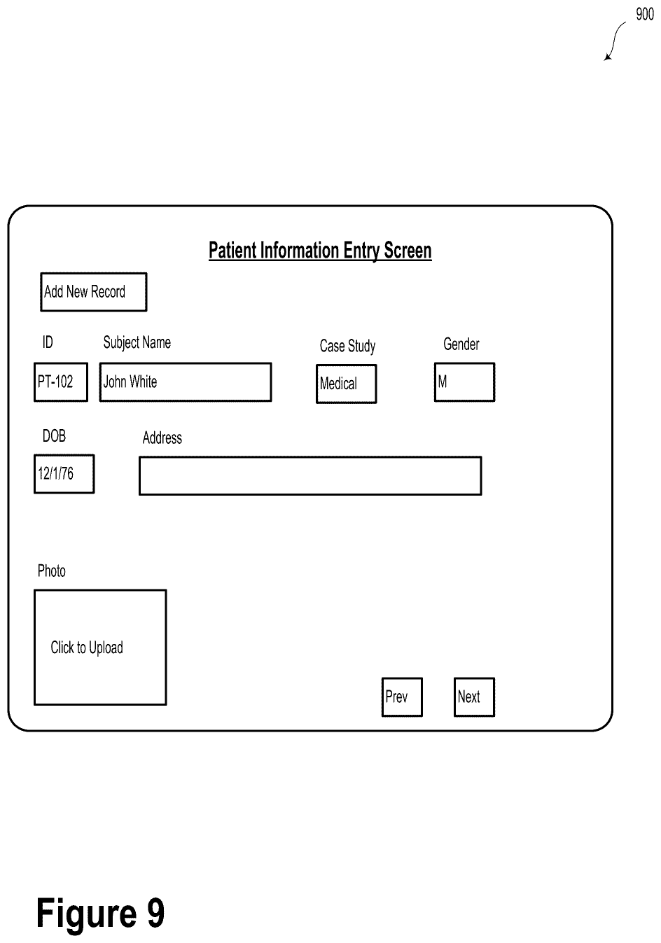

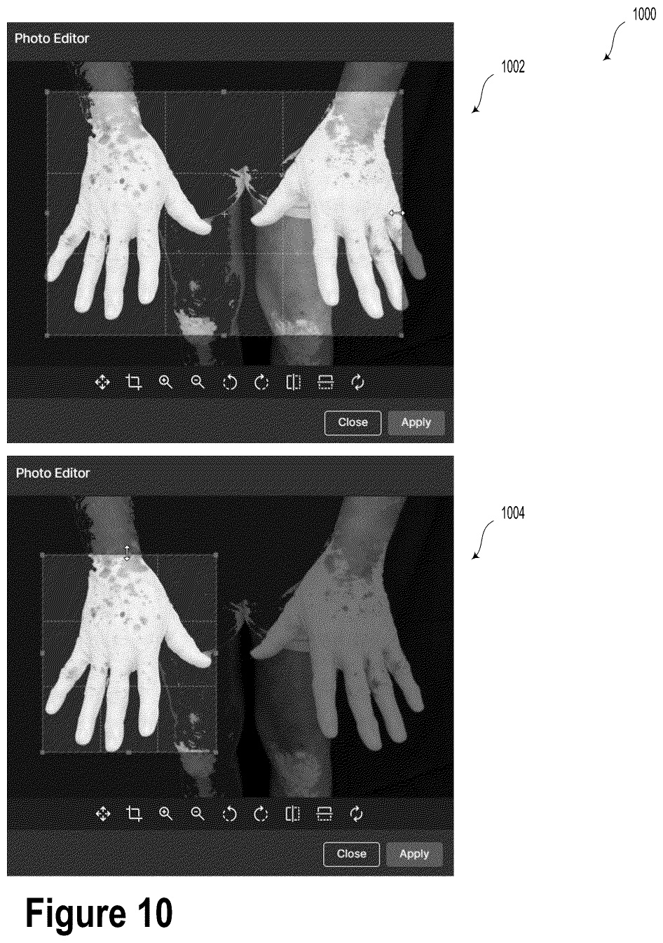

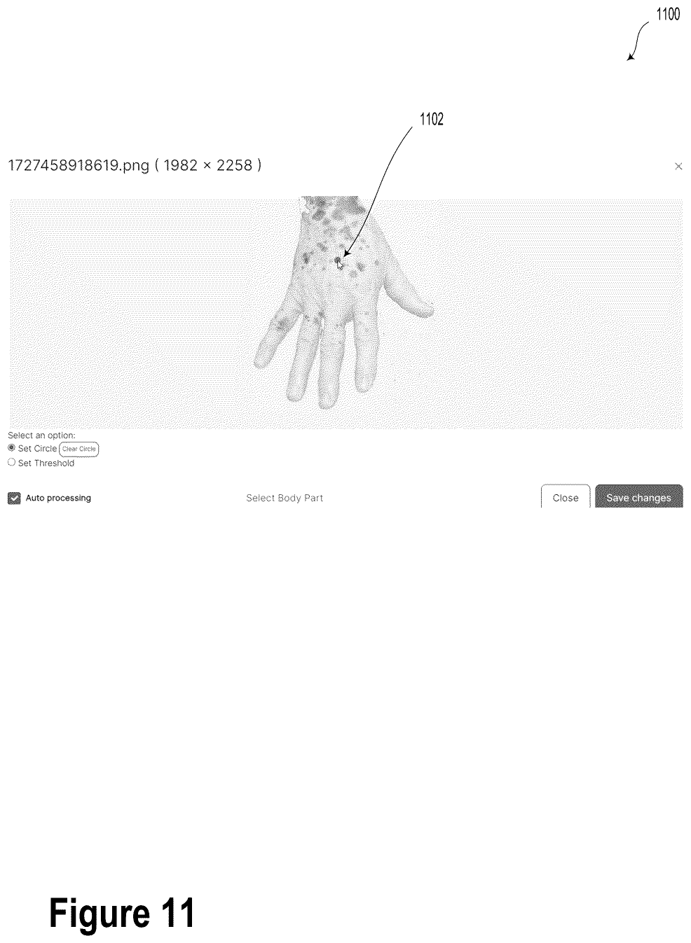

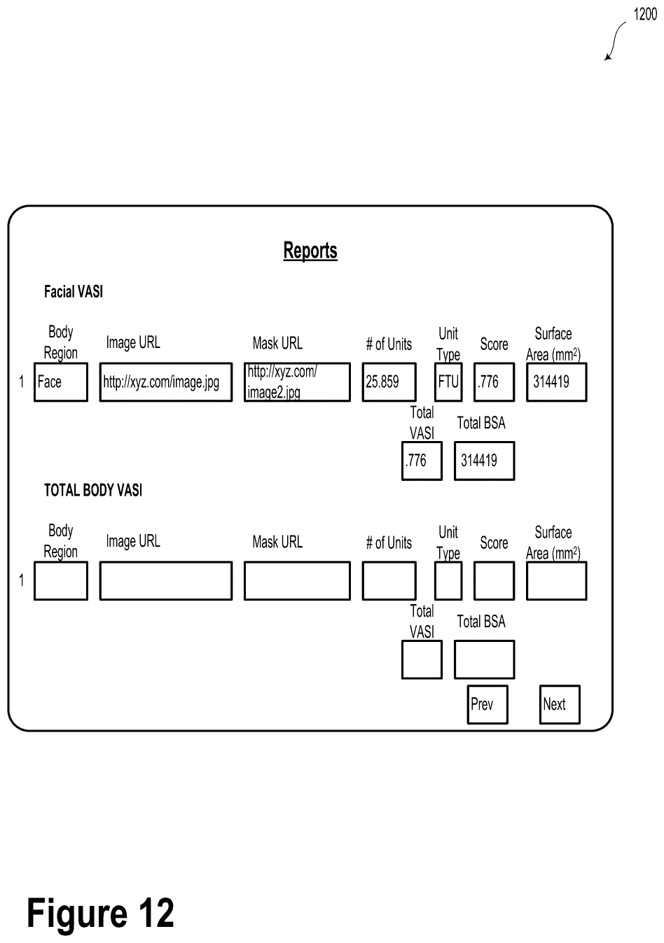

Example embodiments may include a user interface (e.g., graphical user interface 120) that may provide a way for a user to carry out the various image capture and image processing steps described herein. FIGS. 7, 8, 9, 10, 11, and 12 illustrate various displayed screens of a user interface, according to various example embodiment. FIG. 8 includes fictitious patient data for example purposes.

In some examples, the user interface may include a user login screen 700. In such scenarios, the user may be prompted to enter an email address and/or a username and a password. In various examples, users may be able to access individual user accounts. The user accounts could provide various levels of user access privileges. As an example, user access privileges could include administrator, practitioner/doctor, and/or patient.

After logging on, the user may be prompted to review and accept certain terms and conditions of use of the software. In some examples, the user interface may display a patient summary screen. The patient summary screen 800 could display a list of patient records that may include several different fields. As an example, each patient record could include, for example, fields relating to a patient ID, a patient/subject name, a status, date of birth, gender, type of study/study name, and/or a date the patient record was added. The patient summary screen 800 could include a button to add a new patient record. Additionally or alternatively, one or more navigational buttons could be displayed on the user interface. For example, previous and next buttons could allow a user to move between pages of multiple user records.

The user interface may include a patient information entry screen 900. The patient information entry screen could be based on a respective patient record and could accept user entry to add and/or update a patient ID, a patient/subject name, case study type/name, gender, date of birth, address, among other possibilities. In some examples, the case study type/name could include experiment, research, and/or medical, among other examples. The patient information entry screen could include a photo upload field or button. By accessing the photo upload field, a user may be able to add various photographs associated with the patient/subject.

In various examples, uploading and/or interacting with a photo may open a photo editor 1000. The photo editor may display the uploaded photo and allow various photo editing adjustments to be applied such as cropping, rotation, translation, zoom in/out, among other possibilities. In an example embodiment, an uploaded photo of a patient's hands 1002 could be cropped to isolate one of the patient's hands 1004. Once cropped, a masked view 1100 of the cropped photo could be saved as a new image file (e.g., in .png format) and displayed with a white mask background. In the masked view 110, the user could set a graphical circle shape 1102 so as to encircle a calibration dot sticker that had been placed on the patient's hand. In some examples, the user may be able to click and drag the circle shape so as to roughly align with the dot sticker in the masked image. In such scenarios, the image software could be calibrated based on the radius of the circle shape and/or the dot sticker within the image.

In various examples, the user interface could include a pull-down menu, list, or another selection means to select the relevant type of feature (e.g., hand, arm, leg, face, back, torso, etc.) or calibration dot in the image. In some examples, selecting a feature or calibration dot may automatically provide a calibration image, which could include a black and white masked image of the feature. In various examples, multiple images of respective features of a patient/subject could be uploaded, cropped, and/or calibrated in this manner. In some embodiments, a black and white mask could be generated by selecting a threshold value. The threshold value may be adjusted by varying a cluster count slider. In K-means clustering, the cluster count could represent a variable used to partition an image's pixels into k clusters, where k is the cluster count. In such scenarios, each cluster could correspond to a color or intensity in the image. The K-means clustering algorithm assigns each pixel in the image to the closest cluster center (centroid) based on their color values. After clustering, the color of all pixels in one cluster can be replaced by the color of the centroid of that cluster, which is useful for image compression or simplifying the color palette of the image. Additionally or alternatively, the color of respective clusters could be replaced with white or black to form the black and white masked image.

In various embodiments, the user interface could prompt the user to input the dot size diameter in millimeters. In an example embodiment, the user could enter 6.1 mm in the entry window.

In example embodiments, the user interface could include a reports tab 1200, which could be selected to expand a list of spreadsheet-type reports based on the masked images. Each report could provide, for example, information relating to a specific body part (e.g., the patient's face) and/or the user's complete body. The various fields for the specific body part could include, for instance, body region, image URL, mask URL, number of units, unit type (e.g., fingertip unit or FTU), score (per surface area unit), and surface area (e.g., in mm²). Based on the score and surface area, the overall or specific body part VASI score may be automatically calculated.

The particular arrangements shown in the Figures should not be viewed as limiting. It should be understood that other embodiments may include more or less of each element shown in a given Figure. Further, some of the illustrated elements may be combined or omitted. Yet further, an illustrative embodiment may include elements that are not illustrated in the Figures.

A step or block that represents a processing of information can correspond to circuitry that can be configured to perform the specific logical functions of a herein-described method or technique. Alternatively or additionally, a step or block that represents a processing of information can correspond to a module, a segment, or a portion of program code (including related data). The program code can include one or more instructions executable by a processor for implementing specific logical functions or actions in the method or technique. The program code and/or related data can be stored on any type of computer readable medium such as a storage device including a disk, hard drive, or other storage medium.

The computer readable medium can also include non-transitory computer readable media such as computer-readable media that store data for short periods of time like register memory, processor cache, and random access memory (RAM). The computer readable media can also include non-transitory computer readable media that store program code and/or data for longer periods of time. Thus, the computer readable media may include secondary or persistent long term storage, like read only memory (ROM), optical or magnetic disks, compact-disc read only memory (CD-ROM), for example. The computer readable media can also be any other volatile or non-volatile storage systems. A computer readable medium can be considered a computer readable storage medium, for example, or a tangible storage device.

While various examples and embodiments have been disclosed, other examples and embodiments will be apparent to those skilled in the art. The various disclosed examples and embodiments are for purposes of illustration and are not intended to be limiting, with the true scope being indicated by the following claims.

What is claimed is:

1. A system, comprising:
a controller having at least one processor and a memory, wherein the memory stores program instructions that are executable by the at least one processor so as to carry out operations, the operations comprising:
receiving an image of a skin surface;
selecting a combination of color channels from among a plurality of color models;
forming a color-adjusted version of the image based on the selected combination of color channels;
extracting a mask based on the color-adjusted version of the image;
determining, based on the extracted mask, a normal portion of the skin surface;
determining, based on the extracted mask, a differently-pigmented portion of the skin surface; and
providing information indicative of the differently-pigmented portion of the skin surface.

2. The system of claim 1, wherein the selected combination of color channels comprises RGB-B, HSV-V, and Lab-b*.

3. The system of claim 1, wherein the selected combination of color channels are selected from a plurality of color models comprising: RGB (Red, Green, Blue), CMYK (Cyan, Magenta, Yellow, Black), HSV (Hue, Saturation, Value), HSL (Hue, Saturation, Lightness), Lab (Lightness, a*, b*), Lab Color (CIELAB), or XYZ (CIE 1931 Color Space).

4. The system of claim 1, wherein extracting the mask comprises clustering one or more regions of the color-adjusted version of the image so as to form regions of interest.

5. The system of claim 1, wherein extracting the mask comprises utilizing an unsupervised machine learning technique based on color variations of pixels of the color-adjusted version of the image.

6. The system of claim 1, wherein extracting the mask comprises utilizing a trained machine learning model based on color variations of pixels of the color-adjusted version of the image.

7. The system of claim 6, wherein the trained machine learning model was trained with a plurality of training data images.

8. The system of claim 1, wherein providing information indicative of the differently-pigmented portion of the skin surface comprises providing an intelligent-Vitiligo Area Scoring Index (i-VASI) score.

9. The system of claim 1, wherein the image of the skin surface comprises a calibration target, wherein determining the normal portion of the skin surface and determining the differently-pigmented portion of the skin surface is based on an apparent size of the calibration target within the image of the skin surface.

10. The system of claim 1, further comprising:
an image capture apparatus, wherein the operations further comprise:
causing the image capture apparatus to capture the image of the skin surface.

11. The system of claim 1, further comprising:
a graphical user interface (GUI), wherein the operations further comprise:
displaying, via the GUI, an original version of the image and the color-adjusted version of the image; and
displaying the information indicative of the differently-pigmented portion of the skin surface.

12. A method comprising:
providing an image of a skin surface;
selecting a combination of color channels from among a plurality of color models;
forming a color-adjusted version of the image based on the selected combination of color channels;
extracting a mask based on the color-adjusted version of the image;
determining, based on the extracted mask, a normal portion of the skin surface;
determining, based on the extracted mask, a differently-pigmented portion of the skin surface; and
providing information indicative of the differently-pigmented portion of the skin surface.

13. The method of claim 12, wherein the selected combination of color channels comprises RGB-B, HSV-V, and Lab-b*.

14. The method of claim 12, wherein the selected combination of color channels are selected from a plurality of color models comprising: RGB (Red, Green, Blue), CMYK (Cyan, Magenta, Yellow, Black), HSV (Hue, Saturation, Value), HSL (Hue, Saturation, Lightness), Lab (Lightness, a*, b*), Lab Color (CIELAB), or XYZ (CIE 1931 Color Space).

15. The method of claim 12, wherein extracting the mask comprises clustering one or more regions of the color-adjusted version of the image so as to form regions of interest.

16. The method of claim 12, wherein extracting the mask comprises utilizing an unsupervised machine learning technique based on color variations of pixels of the color-adjusted version of the image.

17. The method of claim 12, wherein extracting the mask comprises utilizing a trained machine learning model based on color variations of pixels of the color-adjusted version of the image.

18. The method of claim 17, wherein the trained machine learning model was trained with a plurality of training data images.

19. The method of claim 12, wherein providing information indicative of the differently-pigmented portion of the skin surface comprises providing an intelligent-Vitiligo Area Scoring Index (i-VASI) score.

20. The method of claim 12, wherein the image of the skin surface comprises a calibration target, wherein determining the normal portion of the skin surface and determining the differently-pigmented portion of the skin surface is based on an apparent size of the calibration target within the image of the skin surface.
